(19) United States
(12) Patent Application Publication
(10) Pub. No.: US 2016/0174901 A1
(43) Pub. Date: Jun. 23, 2016

(54) CHILD HEALTH MONITORING TOY

(71) Applicant: ID GUARDIAN LTD., Huddersfield (GB)

(72) Inventor: Josipa Majic, Zagreb (HR)

(21) Appl. No.: 14/971,521

(22) Filed: Dec. 16, 2015

Related U.S. Application Data
(60) Provisional application No. 62/093,989, filed on Dec. 18, 2014.

(51) Int. Cl. A61B 5/00; A61B 5/1455; A63H 3/02; A61B 5/0205

(52) U.S. Cl. CPC A61B 5/6896; A61B 5/02055; A61B 5/14551; A61B 5/742; A61B 5/7405; A63H 3/02; A61B 5/02438

(57) ABSTRACT

An interactive physiological monitoring device includes at least one physiological sensor including an oximetry sensor, at least one motion sensor; and control electronics. The control electronics receive data from the physiological sensor and the motion sensor. The control electronics enter a lower power mode in the absence of a detection of motion from data from the motion sensor over a time interval. The interactive physiological monitoring device further includes a display device, wherein the control electronics cause a representation of data from the physiological sensor to be output to the display device.

### CROSS-REFERENCE TO RELATED PATENT APPLICATIONS

This application claims the benefit of U.S. Provisional Patent Application 62/093,989 filed Dec. 18, 2014 to Majic et al., titled “Child Health Monitoring Toy,” the contents of which are incorporated herein by reference in their entirety.

### BACKGROUND

When a person visits a doctor or hospital, the unfamiliar environment and unfamiliar equipment can lead to emotional stress and an unwillingness to cooperate with a healthcare professional. It can be difficult and time consuming to get a person (e.g., a child, elderly person, or other person) to sit still or relax to measure vital signs. In addition, stress may cause the person's vital signs to change, which can lead to skewed medical data and a potentially unreliable picture of the person's current health condition.

It would therefore be desirable to have available a measurement device that is familiar and non-threatening, to minimize the stress in a healthcare environment. Such a device would further be beneficial for monitoring within a home environment.

### SUMMARY

An interactive physiological monitoring device includes at least one physiological sensor including an oximetry sensor, at least one motion sensor; and control electronics. The control electronics receive data from the physiological sensor and the motion sensor. The control electronics enter a lower power mode in the absence of a detection of motion from data from the motion sensor over a time interval. The interactive physiological monitoring device further includes a display device, wherein the control electronics cause a representation of data from the physiological sensor to be output to the display device.

An interactive physiological monitoring system includes at least one physiological monitoring device, and instructions stored in a non-transitory computer-readable storage medium for execution by a processor, including instructions for receiving information from the physiological monitoring device, analyzing the received information, and providing display data representing one of the analyzed information and the received information from the physiological monitoring device. The physiological monitoring device includes at least one physiological sensor, including an oximetry sensor. The physiological monitoring device further includes at least one motion sensor, and control electronics configured to receive data from the physiological sensor and the motion sensor, wherein the control electronics are further configured to enter a lower power mode in the absence of a detection of motion for a time interval.

A method includes receiving a pulse measurement from a heart rate sensor in a toy, providing to a display in the toy a representation of the heart rate measurement, and storing information related to the pulse measurement in a memory in the toy.

### BRIEF DESCRIPTION OF THE DRAWINGS

FIG. 1 is a block diagram representing an object incorporating a monitoring system according to an embodiment of the present disclosure.

FIG. 2 is an illustration of an object incorporating a monitoring system according to an embodiment of the present disclosure.

FIG. 3A is an illustration of a portion of an object incorporating a monitoring system according to an embodiment of the present disclosure.

FIG. 3B is an illustration of an object incorporating a monitoring system according to an embodiment of the present disclosure.

FIG. 4 illustrates a portion of the design and testing performed to implement a marketable product according to an embodiment of the present disclosure.

FIG. 5 is a schematic of a monitoring system according to an embodiment of the present disclosure.

FIGS. 6A, 6B, 6C are examples of housing for a monitoring system according to an embodiment of the present disclosure.

FIG. 7 is an illustration of an object incorporating a monitoring system according to an embodiment of the present disclosure.

FIGS. 8A and 8B illustrate displays at a graphical interface user interface according to an embodiment of the present disclosure.

FIG. 9 illustrates an example of a visual parameter mapping at a graphical interface user interface according to an embodiment of the present disclosure.

FIG. 10 illustrates an example of a visual parameter mapping at a graphical interface user interface according to an embodiment of the present disclosure.

FIG. 11 an example of an environment equipped with monitoring systems according to embodiments of the present disclosure.

FIG. 12 an example of an environment equipped with monitoring systems according to embodiments of the present disclosure.

### DETAILED DESCRIPTION

A monitoring system is incorporated into an object that is non-threatening. For example, the monitoring system may be incorporated into a toy. One embodiment discussed in the present disclosure by way of example is a stuffed animal in which a monitoring system is incorporated. Stuffed animals (or other toys, or other objects) may serve to reduce stress in a child, and may also serve to reduce stress in an adult. Thus, although portions of the present disclosure may be discussed in relation to a child, it should be understood that the monitoring system of the present disclosure is equally applicable for the monitoring of adults.

The monitoring system includes one or more sensors to measure physiological signals, and may include one or more sensors to monitor motion. Sensor information may be evaluated as a single measurement (e.g., temperature) or as a time series of measurements. By tracking sensor data over time, trends in health and activity may be identified, or health conditions may be recognized. Thus, data from sensors can be used to determine physiological state (e.g., illness, high or increasing blood pressure, low or decreasing blood oxygen level, or irregular heart beat pattern or rate) and activity state (e.g., restlessness, lethargy, walking, jumping, or rocking side to side).

Data from the sensors may be used to present a visual display on the object incorporating the monitoring system. Information (e.g., sensor data, physiological state, or activity state) may be provided via a communication interface from the monitoring system to an external device for storage, analysis, or display. The external device may be remote, such as for monitoring a person from a monitoring center or health care provider facility. Alternatively, an intermediate device receives information from the object and provides information to a remote device. Remote monitoring allows for fewer visits of a person to the healthcare facility, which also reduces exposure of the person to other illnesses.

The monitoring system provides for interactive and non-threatening monitoring of a person. Interactivity includes providing information regarding the monitored person at one or both of a display interface of an object incorporating the monitoring system, or at a display on an external device. For example, a person can see a visualization of their heart beat and calm themselves to slow the heartbeat, or see a visualization of blood oxygen level and perform an activity to increase blood oxygen level. For another example, a caregiver can see a visualization of irregular heart beat or heart rate and notify a healthcare provider, or see a visualization in trends in activity level and encourage the monitored person to be more active.

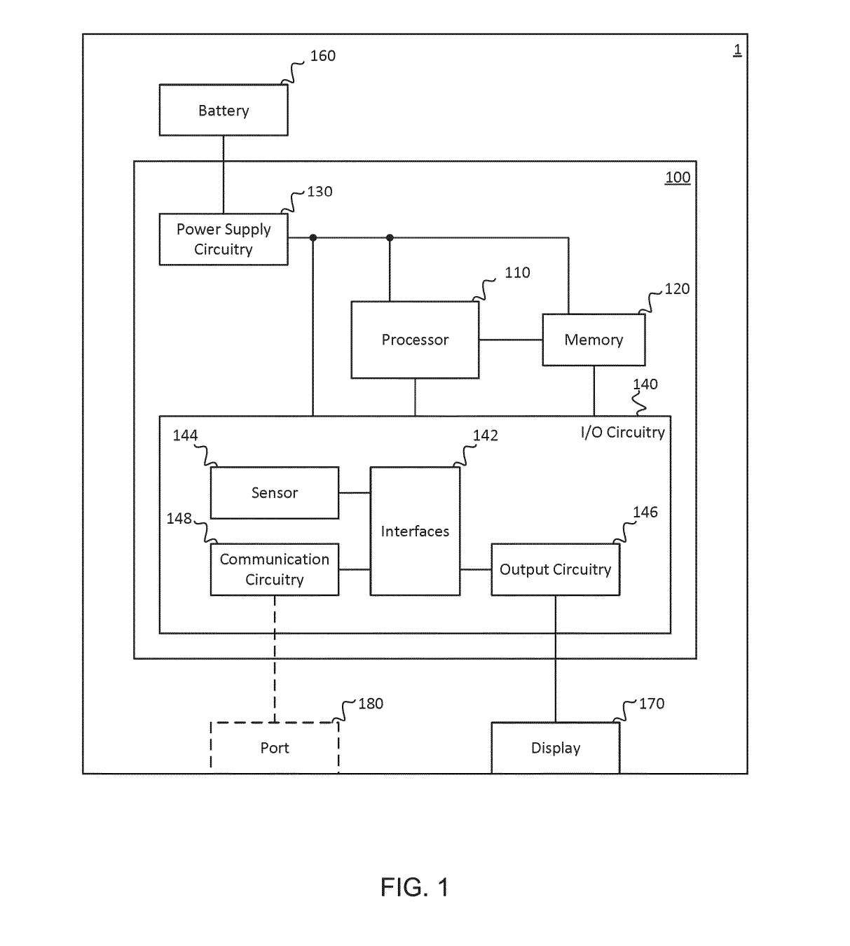

FIG. 1 is a block diagram representing an embodiment of an object 1 incorporating a monitoring system 100 according to an embodiment of the present disclosure. The monitoring system 100 is implemented as control electronics including a processor 110, a memory 120, power supply circuitry 130, and input/output (I/O) circuitry 140.

Processor 110 represents a programmable processor, which may be, for example, a general-purpose processor, digital signal processor, microprocessor, microcontroller, application specific integrated circuit (ASIC), field programmable gate array (FPGA), other circuitry effecting processor functionality, or multiple ones or combinations of the foregoing, along with associated logic and interface circuitry. Processor 110 may be incorporated in a system on a chip.

Memory 120 represents one or both of volatile and non-volatile memory for storing information (e.g., instructions and data). Examples of memory include semiconductor memory devices such as EPROM, EEPROM, flash memory, RAM, or ROM devices.

Portions of monitoring system 100 may be implemented as computer-readable instructions in memory 120, executed by processor 110.

An embodiment of the disclosure relates to a non-transitory computer-readable storage medium (e.g., memory 120) having computer code thereon for performing various computer-implemented operations. The term “computer-readable storage medium” is used herein to include any medium that is capable of storing or encoding a sequence of instructions or computer codes for performing the operations, methodologies, and techniques described herein. The media and computer code may be those specially designed and constructed for the purposes of the embodiments of the disclosure, or they may be of the kind well known and available to those having skill in the computer software arts.

Examples of computer code include machine code, such as produced by a compiler, and files containing higher-level code that are executed by a computer using an interpreter or a compiler. For example, an embodiment of the disclosure may be implemented using Java, C++, or other object-oriented programming language and development tools. Additional examples of computer code include encrypted code and compressed code. Moreover, an embodiment of the disclosure may be downloaded as a computer program product, which may be transferred from a remote computer (e.g., a server computer) to a requesting computer (e.g., a client computer or a different server computer) via a transmission channel. Another embodiment of the disclosure may be implemented in hardwired circuitry in place of, or in combination with, machine-executable software instructions.

Power supply circuitry 130 distributes electrical power to components of monitoring system 100. Power supply circuitry 130 may include, for example, a power supply semiconductor device (e.g., a voltage regulator) with related configuration components and external filters. Power supply circuitry 130 may distribute power over multiple paths, where the power distributed in each path may have similar or different power ratings (e.g., voltage level or current level).

I/O circuitry 140 represents electrical components and optional code that together provide monitoring system 100 access to an environment external to monitoring system 100. For example, I/O circuitry 140 may provide access to components of object 1, to a person handling object 1, or to a device communicating with object 1. I/O circuitry 140 includes one or more interfaces 142, one or more sensors 144, output circuitry 146, and communication circuitry 148.

Interfaces 142 represent electrical components and optional code, such as programmable integrated circuits, non-programmable integrated circuits, filtering components, level shifters, analog-to-digital or digital-to-analog converters, and other components, with associated electrical connections (e.g., wiring or traces, or connectors). Interfaces 142 provide electrical pathways and/or communication pathways between components of monitoring system 100. For example, one or more interfaces 142 may be provided between sensor 144 and communication circuitry 148, between communication circuitry 148 and output circuitry 146, or between sensor 144 and output circuitry 146. For another example, one or more interfaces 142 may be provided between processor 110 and sensor 144, output circuitry 146, or communication circuitry 148. For a further example, one or more interfaces 142 may be provided between memory 120 and sensor 144, output circuitry 146, or communication circuitry 148, for direct transfer of data to or from memory 120.

Sensor 144 detects a physiological signal or a motion signal. Multiple sensors 144 may be included in monitoring system 100.

Sensors 144 may measure a wide range of vital signs, such as temperature, heart rate, heart rate variability, respiratory rate, blood oxygen level, humidity, blood pressure, chemicals present, or the like. Further, sensors 144 may be placed to monitor an environment, such as noise sensors, temperature sensors, humidity sensors, light sensors and air quality sensors. Environment data may be used to adjust or analyze data from physiological sensors 144. For example, if there is a high ambient temperature, a person's slightly higher-than-normal temperature may be disregarded in some instances. Sensors 144 may also be used to monitor the electronics of monitoring system 100, such as a temperature sensor within a sensor housing or control electronics housing to identify and respond to overheating of the electronics, where a response may include reduced functionality, error messages transmitted, audiovisual warnings, or shutdown of the electronics.

Sensor 144 may monitor an aspect of position or motion. Examples include an accelerometer or other motion detector, a relative position sensor, a global positioning sensor, or a proximity sensor. Information from a motion detector may be used, for example, to gauge a child's activity level, to modify or disregard measurements from other sensors (for example, if movement would render the measurements questionable or invalid), and to identify if the electronics can be transitioned to a lower power level (such as by switching off circuits and/or functionality) in the absence of a detection of motion over a time interval.

Data from sensor 144 may be stored in memory 120 for later analysis. Additionally or alternatively, raw or filtered data may be provided externally via communication circuitry 148, and/or data may be analyzed by processor 110 and the analyzed data stored in memory 120 or provided externally.

Communication circuitry 148 represents electrical components and optional code that together provide an interface from internal components of monitoring system 100 to an external network. For example, communication circuitry 148 may be a Bluetooth protocol physical layer circuit with associated software protocol layers, a Wi-Fi protocol physical layer circuit with associated software protocol layers, an Internet protocol physical layer circuit with associated software protocol layers, or other standard or proprietary circuit and software. Communication circuitry 148 may communicate bi-directionally, such that, for example, data may be sent from monitoring system 100, and instructions and updates may be received by monitoring system 100. Communication externally may be, for example, with a computing device, such as a desktop, laptop, set-top box, or mobile computer (i.e., smart phone or tablet or the like), or such as a hub device which gathers information from several devices, or such as a remote server.

Portions of monitoring system 100 may be integrated together. For example, memory 120 may be integrated with processor 110, portions of I/O circuitry 140 may be integrated with processor 110, communication circuitry 148 may be integrated with an interface 142 and processor 110, or other integrations. Thus, the blocks of FIG. 1 represent groupings of circuitry by function, and illustration of circuitry as being in different blocks does not necessarily represent (although it can) a corresponding physical separation between physical components.

Object 1 includes a battery 160 to provide power to monitoring system 100. Object 1 further includes an optional display 170 coupled to output circuitry 146. In one or more embodiments, a representation of data received from sensor 144 is provided to display 170 (e.g., on a graphical user interface of display 170). In one or more embodiments, a representation of health state, physiological state, or activity state is provided to display 170.

Object 1 may optionally include a port 180 coupled to communication circuitry 148 for physical connection to an external device, such as through a wired link to an external computer.

Having described generally an embodiment of monitoring system 100 incorporated into an object 1, next is described an example in which object 1 is implemented as a Teddy the Guardian (“Teddy”) toy bear. It is to be understood that other stuffed animals, other toys and other objects are encompassed by this disclosure.

Teddy is a plush teddy bear designed to be safe, easy to use, and easy to maintain. Information obtained by Teddy may be presented in an easy to understand visual display. Teddy includes an ability to create multiple profiles, log health metrics from other devices, log drugs administered, and so forth, to keep a detailed diary for a person or multiple persons.

The Teddy product has a removable soft flexible outer shell that is washable. The electronics are secured within a hidden inner shell. The electronics are designed to have low power consumption. A battery (e.g., battery 160) is positioned within the inner shell.

Teddy includes built-in medical sensors (e.g., sensor 144) for monitoring vital signs and motion sensors (e.g., sensor 144) for monitoring position or movement. Information may be provided externally by way of a wired or wireless communication interface (e.g., via communication interface 148, or via communication interface 148 and port 180). Additionally or alternatively, Teddy may store data in an internal memory (e.g., memory 120).

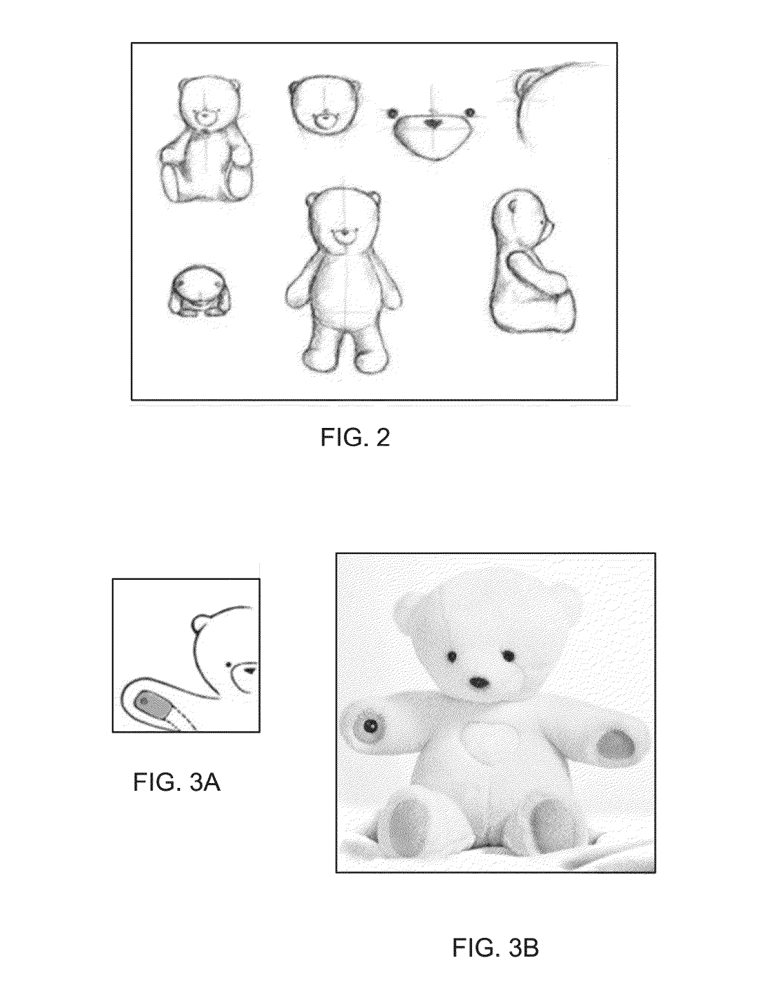

FIG. 2 illustrates, by way of introduction to Teddy, drawings of Teddy according to an embodiment of the present disclosure, where Teddy is illustrated from various angles and with enlargements of some features.

FIG. 3A illustrates a head and paw portion of Teddy, showing placement of a monitoring system 100 in Teddy's paw, also showing in dotted line a placement path of how the monitoring system 100 is positioned within Teddy's paw.

FIG. 3B is an image of an embodiment of Teddy, with monitoring system 100 positioned in Teddy's paw. In the embodiment of FIG. 3B, a temperature sensor is exposed on an inner side of the paw. When a person holds Teddy's paw, or the paw is placed on a person's forehead, the temperature sensor may take measurements. The temperature sensor may be infrared, so that the sensor may also measure temperature without direct contact, such as when the bear is being hugged or otherwise held. In one or more embodiments, a pulse oximetry sensor is positioned on the outside of the paw so that a person may hold Teddy by the paw to facilitate measurement. Alternatively or additionally to temperature and pulse oximetry sensors, other sensors (e.g., sensors 144) may be used in this embodiment.

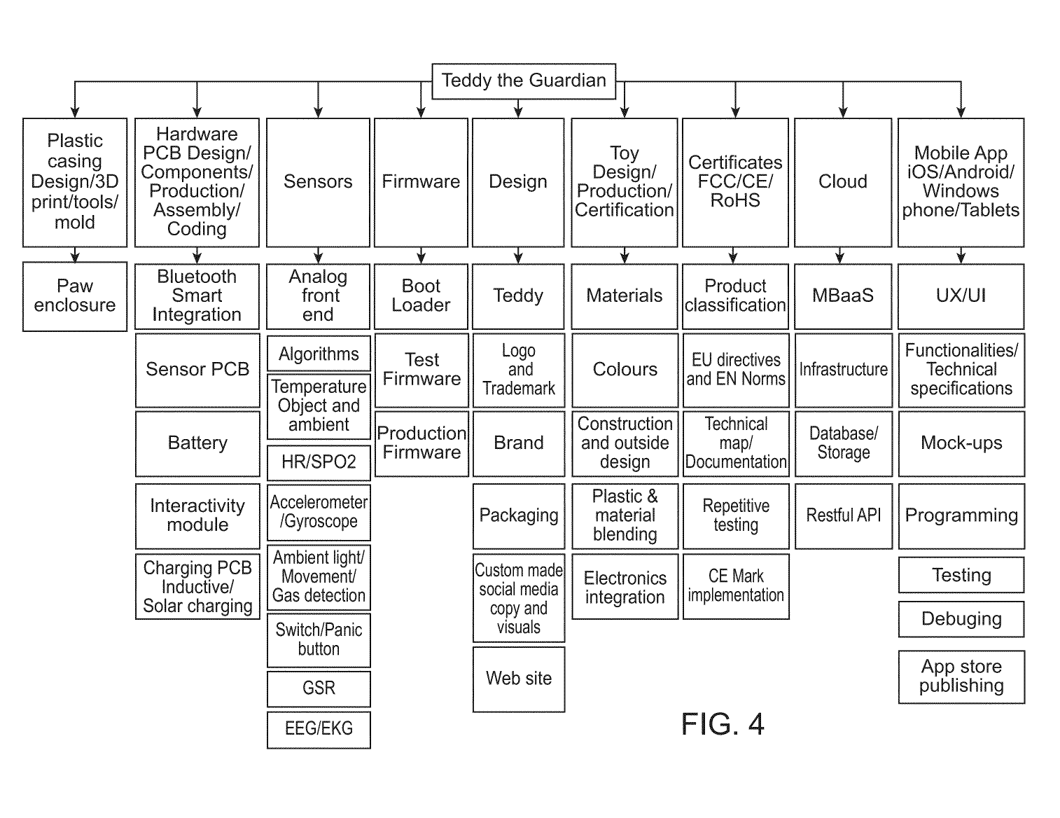

FIG. 4 illustrates a portion of the design and testing performed to implement a marketable Teddy product.

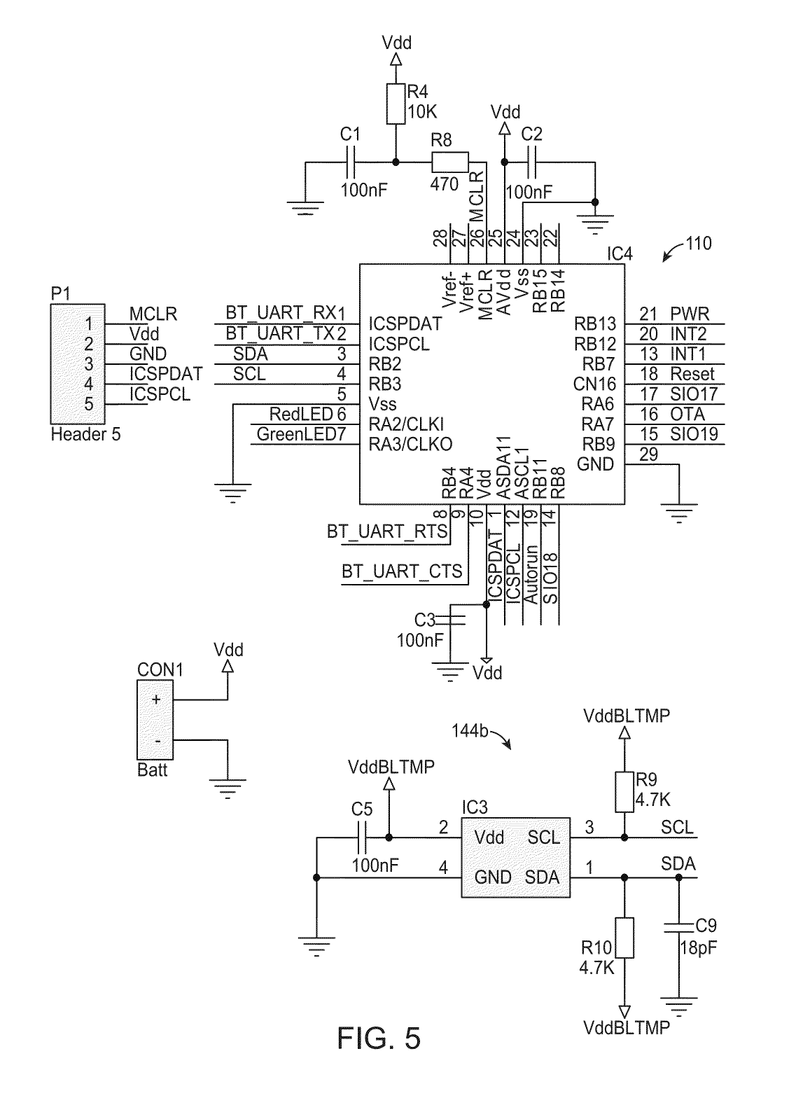

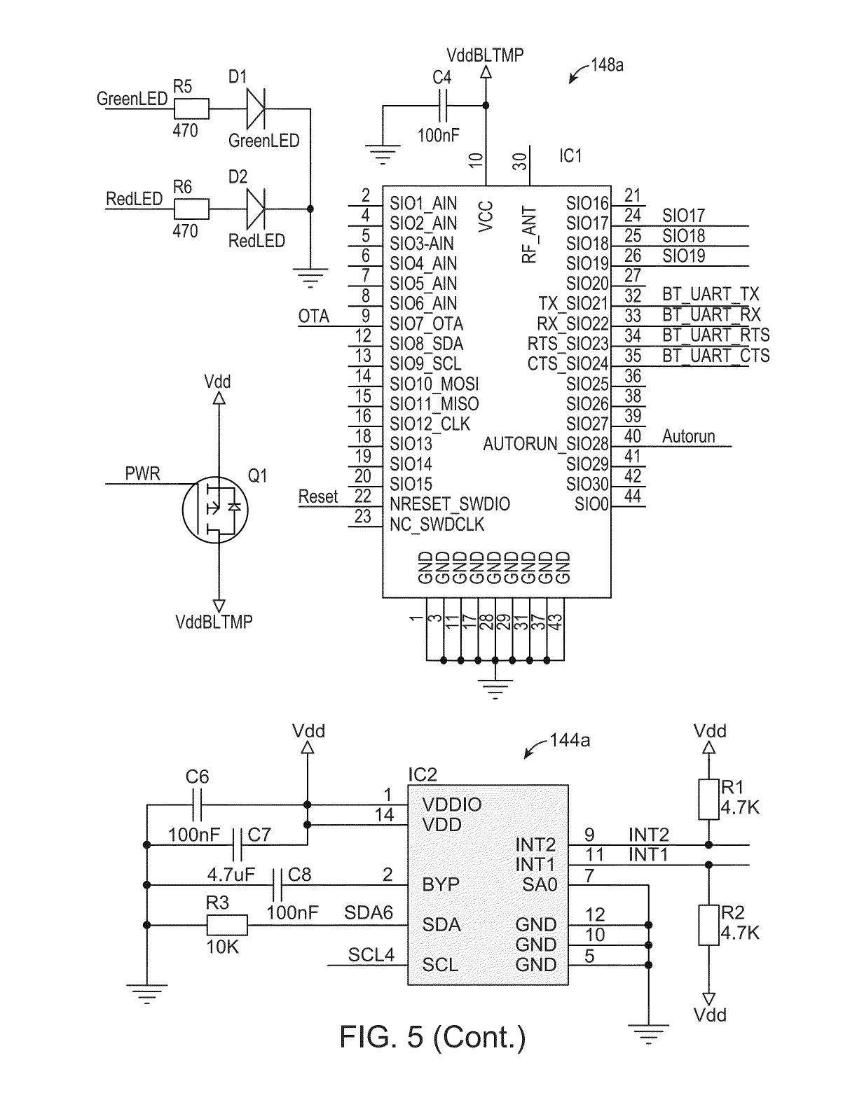

FIG. 5 is a schematic of an embodiment of monitoring system 100 incorporated into the Teddy embodiment illustrated in FIG. 3B. In this embodiment of monitoring system 100, processor 110 is a Microchip Technology Inc. processor PIC24F32KA302 with built-in memory; sensors 144 include a Freescale Semiconductor, Inc. MMA8451Q accelerometer (sensor 144a), a Melexis Semiconductors MLX90615 infrared sensor (sensor 144b), and an APM Korea DCM03 touch sensor with a Texas Instruments Inc. AFE4490 analog front end (not shown); and communications circuitry 148 includes a Laird Technologies, Inc. BL6000 Bluetooth module (148a).

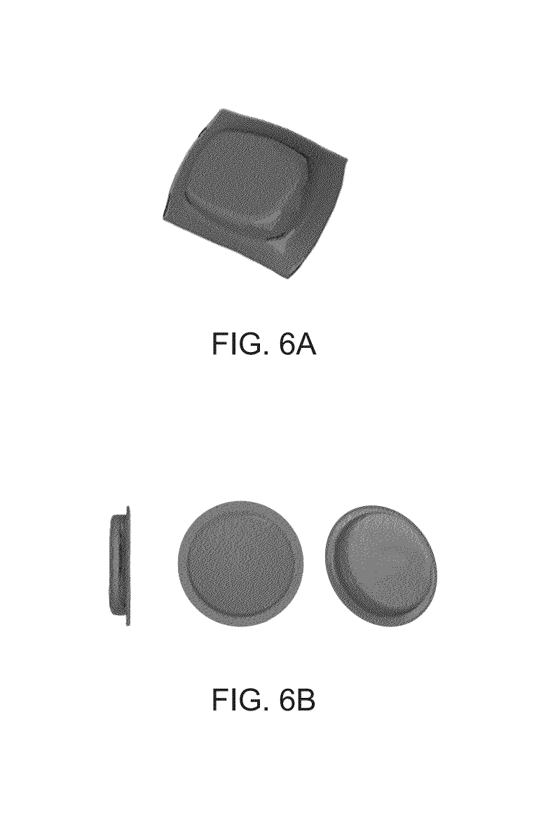

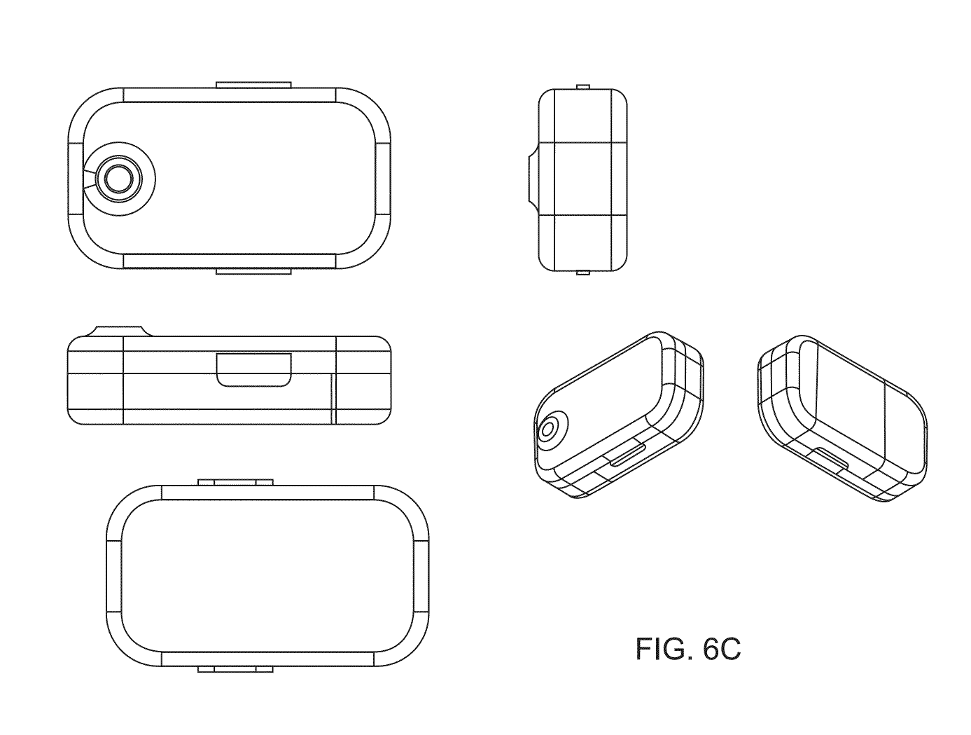

FIG. 6A-6C illustrate examples of housings (the hidden inner shell) in which monitoring system 100 may be incorporated. A housing may be formed, for example, by injection molding or three-dimensional printing. FIG. 6A illustrates a sealed rectangular package, FIG. 6B illustrates a sealed circular package, and FIG. 6C illustrates in detail a two-piece rectangular package that is snap-fitted together. A gasket may optionally be provided between the two pieces to provide a seal, or other sealing technique may optionally be used.

In the embodiment of FIG. 5, monitoring system 100 is implemented within a single housing. In other embodiments, one or more components of monitoring system 100 may be implemented in separate housings. For example, temperature sensor 144 may be positioned at one location in object 1 (e.g., in Teddy's forehead), pulse oximeter sensor 144 may be positioned at another location in object 1 (e.g., in Teddy's paw), and portions of the remainder of monitoring system 100 may be positioned in yet another location in object 1 (e.g., with a display 170).

In or more embodiments of the present disclosure, a housing or housings may be removed from Teddy or other object 1, for cleaning object 1 and the housing separately.

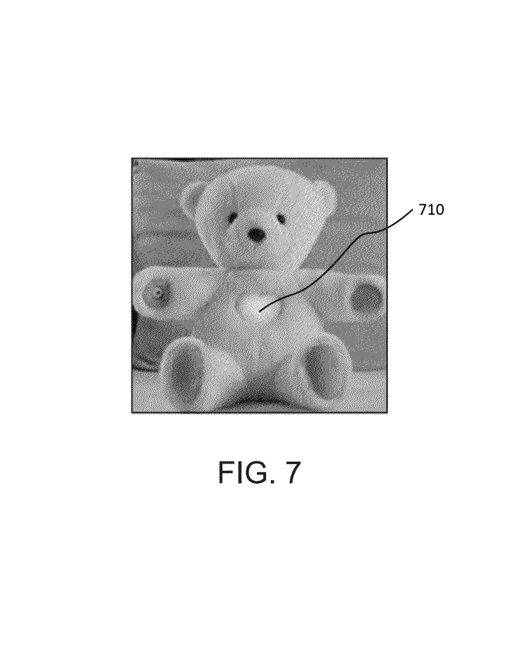

FIG. 7 illustrates an embodiment of Teddy which includes interaction functionality. As illustrated in FIG. 7, a heart shaped visual indicator 710 (e.g., display 170) is affixed to or incorporated within Teddy's torso. Visual indicator 710 uses light, light intensity and color to interact with a person, and also may include audio interaction. Visual indicator 710 may provide one or more lights continuously or in sequence to provide a comforting or calming environment. Music or other comforting or calming sounds may be provided additionally or alternatively.

Visual indicator 710 may provide visual or audio indications of a person's physiological or activity state. For example, visual indicator 710 may provide pulsed light or sound corresponding to a heartbeat, or colored light indicating temperature. Visual indicator 710 may include a capability to display images or text. In some embodiments, sequences to be provided at visual indicator 710 may be received from an external computing device. For example, the external computing device may transmit information or images to processor 110 for creating a display (or audio sequence) at visual indicator 710.

The external computing device may also provide visual feedback. For example, for a given parameter such as temperature, heart rate, or oxygen level, parameter information may be displayed as a present value, a history of values, or a change from an average value. Parameter information may be displayed in relation to an expected range, or in comparison to a threshold value, such as to indicate that the monitored person should be taken for examination by a healthcare professional. Other display options will be readily apparent to one skilled in the art.

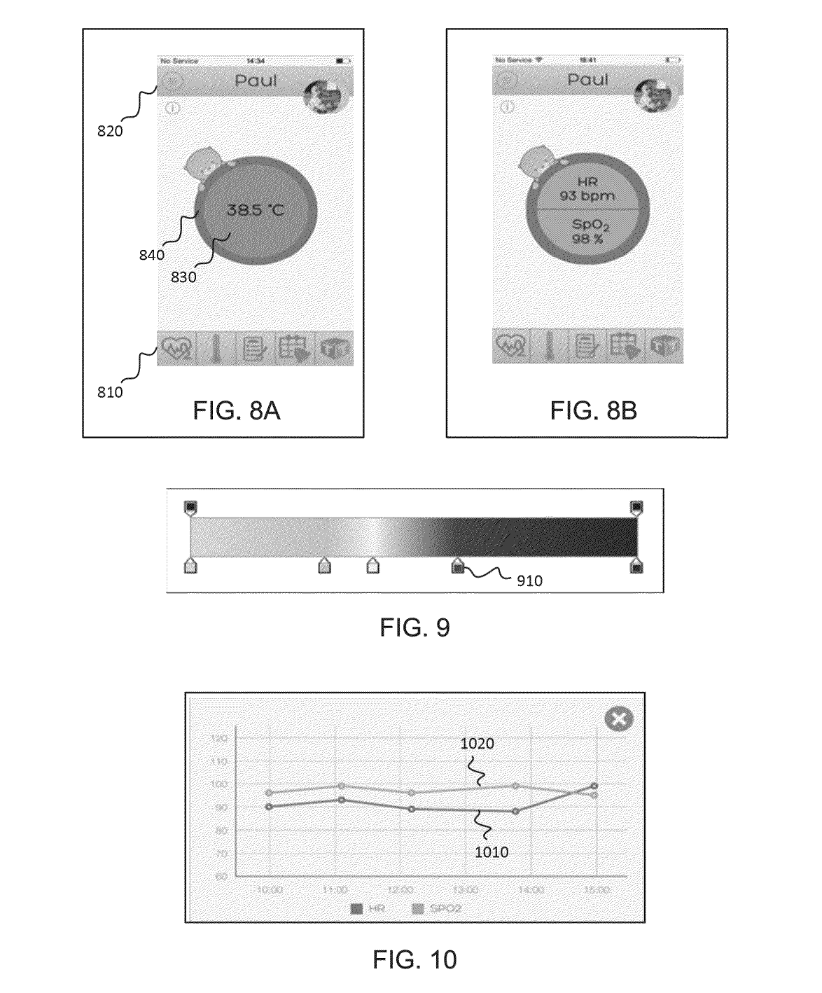

FIGS. 8A and 8B illustrate an example of a graphical user interface (GUI) of an external computing device, which is shown as providing information for child ‘Paul’. As illustrated for this example, a user may select from selector bar 810 to view information related to various sensors, to view history data, to set up and acknowledge dosing schedules and reminders, and to select information to access or review from monitoring systems 100 of other objects 1. The user may select from selector bar 820 to see information related to a different person.

In the example of FIG. 8A, a temperature measurement of “38.5° C.” is displayed within an area 830. User settings allow for display in degrees Celsius (° C.) or degrees Fahrenheit (° F.). In some embodiments, display color (or percent gray on the grayscale) within area 830 or at a perimeter 840 of area 830 is related to temperature measurement through a mapping.

In the example of FIG. 8B, a heart rate and blood oxygen level are numerically displayed. In some embodiments, display color (or percent gray on the grayscale) within area 830 or at a perimeter 840 of area 830 is related to heart rate or blood oxygen level through a mapping. A visual indicator may pulsate in synchrony with a sensed heartbeat, such as inside the area 830 or along perimeter 840 of area 830 in FIG. 8B. Pulsating may be visually indicated by, for example, the use of alternating colors, or by colors appearing and disappearing.

FIG. 9 illustrates an example of a visual parameter mapping in grayscale. For example, the grayscale mapping may represent measured temperature in a range of 35.0° C. at the left of the mapping to 41.9° C. at the right of the mapping. Markers may be used (such as markers 910 in FIG. 9) to indicate thresholds, such as when to contact a healthcare provider, or markers may be used to indicate daily, weekly and monthly averages.

Other sensed parameters may also be presented by way of a mapping. Color or grayscale mappings may be presented in many forms, such as the linear left-to-right mapping in FIG. 9, a target-style mapping of concentric circles, or other form. Other mappings may be used, such as indicating pulse rate by a density of dots or lines. Various types of markers may be implemented.

A mapping may be saved in a memory, such as in an equation or as a table of values. For example, Table 1 may be used to map a temperature range of 35.0° C. to 41.3° C. to a small number of colors for display.

TABLE 1

| Color | ICE BLUE | GREEN | YELLOW | RED |
|---|---|---|---|---|
| Temp. ° C. | 35.0-36.9 | 37.0-37.7 | 37.8-39.0 | 39.1-41.3 |

The same color mapping, or a different color mapping, may be used to indicate temperature in the visual indicator built into the object 1.

FIG. 10 illustrates an example of a visual presentation on the GUI of the external computing device, showing heart rate (line 1010) and oxygen level (line 1020) measurement history.

As noted above, the monitoring system 100 may be incorporated into a variety of objects 1. Further, multiple objects 1 may be used, for example, to establish a larger monitored space, to incorporate different types of sensors 144 into different objects 1 within a monitored space, to monitor behavior of a person as they move between objects 1 in a monitored space, to monitor interaction of a person with one or more objects 1 in a monitored space, or to monitor multiple persons concurrently.

For example, multiple objects 1 within a room may be activated based on one or more of proximity, motion or temperature, and then may record sensed data, analyze sensed data, provide raw, filtered, or analyzed data externally, and display visual indications related to the sensed data, as described above. An example of a discreet object 1 is a patch including one or more sensors and a communication interface, positioned on furniture, on a wall, on a baby bed, and so forth. Object 1 may in the form of a wearable item. The various objects 1 may communicate with each other, and may communicate with a hub (or an object 1 designated as a hub). Hub devices may collect data from multiple objects 1 to track a person's physiological state and activity state even as the child moves between toys and areas in the environment. Visual indicators may, in some embodiments, be placed around the environment, such as in wall hangings, ornaments, lamps, or display devices.

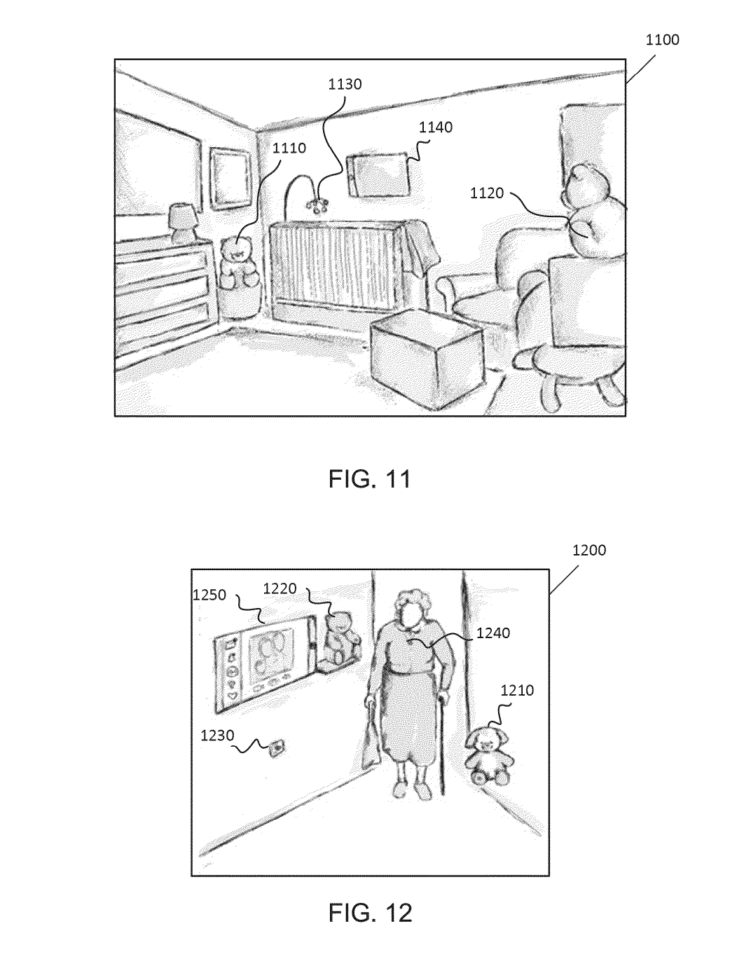

FIGS. 11 and 12 illustrate two examples of environments in which multiple objects 1 are incorporated. It should be understood that many other types of environments are also envisioned, and an environment may include one or more objects 1. For example, an environment may be a doctor's office with one Teddy, or with multiple objects 1 each assigned to a different person. Other examples of environments include a living room, a play room, a bedroom, a hospital room, a daycare facility room, and so forth.

FIG. 11 illustrates an example of a nursery 1100 equipped with multiple objects 1, suitable for use around children. Examples include Teddy 1110, Teddy 1120, a mobile 1130 on the baby bed, a display and control device 1140 on the wall, among others.

FIG. 12 illustrates an example of space 1200 equipped with multiple objects 1, possibly more suitable for adults. Note that Teddy or other stuffed animals are illustrated in the space in FIG. 12, indicating that stuffed animals may be suitable for persons of any age. Note also that an object 1 in a bunny 1210 form includes a sensor in a hind paw. Examples of objects 1 in space 1200 also include Teddy 1220, a sensor 1230 attached on the wall, a sensor 1240 as a wearable (necklace), and a display and control device 1250 on the wall.

By analyzing data from multiple sensors, an emotional state of a person may also be determined.

While the disclosure has been described with reference to the specific embodiments thereof, it should be understood by those skilled in the art that various changes may be made and equivalents may be substituted without departing from the true spirit and scope of the disclosure as defined by the appended claims. In addition, many modifications may be made to adapt a particular situation, material, composition of matter, method, operation or operations, to the objective, spirit and scope of the disclosure. All such modifications are intended to be within the scope of the claims appended hereto. In particular, while certain methods may have been described with reference to particular operations performed in a particular order, it will be understood that these operations may be combined, sub-divided, or re-ordered to form an equivalent method without departing from the teachings of the disclosure. Accordingly, unless specifically indicated herein, the order and grouping of the operations is not a limitation of the disclosure.

What is claimed is:

1. An interactive physiological monitoring device, comprising:
at least one physiological sensor, the at least one physiological sensor including an oximetry sensor;
at least one motion sensor;
control electronics configured to receive data from the at least one physiological sensor and the at least one motion sensor, wherein the control electronics are further configured to enter a lower power mode in the absence of a detection of motion from data from the at least one motion sensor over a time interval; and
a display device, wherein the control electronics are further configured to cause a representation of data from the at least one physiological sensor to be output to the display device.

2. The monitoring device of claim 1, implemented as a toy.

3. The monitoring device of claim 2, wherein the toy is a stuffed animal, and the at least one physiological sensor is positioned in an appendage of the stuffed animal.

4. The monitoring device of claim 1, wherein the at least one physiological sensor includes a temperature sensor.

5. The monitoring device of claim 4, wherein the representation of data output to the display device causes the display to produce a color representing a temperature determined from data from the temperature sensor.

6. The monitoring device of claim 1, wherein the control electronics are further configured to determine an activity level of a subject based on data from the at least one motion sensor.

7. The monitoring device of claim 1, wherein the at least one motion sensor includes an accelerometer.

8. The monitoring device of claim 1, further comprising a proximity sensor.

9. The monitoring device of claim 1, further comprising a communication interface for providing information based on the data from the at least one physiological sensor and the at least one motion sensor to an external device.

10. The monitoring device of claim 1, wherein the representation of data output to the display device causes the display to pulse varied colors in synchrony with a heartbeat detected by way of the at least one physiological sensor.

11. The monitoring device of claim 1, wherein data from the at least one physiological sensor includes at least two of temperature, heart rate, heart rate variability, respiratory rate, blood oxygen level, humidity, blood pressure, and chemicals present.

12. The monitoring device of claim 1, further comprising at least one of an ambient temperature sensor, an ambient noise sensor, an ambient humidity sensor, an ambient light sensor and an ambient air quality sensor.

13. An interactive physiological monitoring system, comprising:
at least one physiological monitoring device; and
instructions stored in a non-transitory computer-readable storage medium for execution by a processor, including instructions for receiving information from the at least one physiological monitoring device, analyzing the received information, and providing display data representing one of the analyzed information and the received information from the at least one physiological monitoring device;
wherein the at least one physiological monitoring device includes:
at least one physiological sensor, the at least one physiological sensor including an oximetry sensor;
at least one motion sensor; and
control electronics configured to receive data from the at least one physiological sensor and the at least one motion sensor, wherein the control electronics are further configured to enter a lower power mode in the absence of a detection of motion for a time interval.

14. The monitoring system of claim 13, wherein the at least one physiological monitoring device is a plurality of monitoring devices positioned in variable locations in an environment.

15. The monitoring system of claim 14, wherein the instructions further include instructions for analyzing the received information from the plurality of monitoring devices and identifying a physiological state of a subject in the environment.

16. The monitoring system of claim 14, wherein the instructions further include instructions for analyzing the received information from the plurality of monitoring devices and identifying an emotional state of a subject in the environment.

17. The monitoring system of claim 14, wherein the instructions further include instructions for analyzing the received information from the plurality of monitoring devices and identifying an activity state of a subject in the environment.

18. A method, comprising:
receiving a pulse measurement from a heart rate sensor in a toy;
providing to a display in the toy a representation of the heart rate measurement; and
storing information related to the pulse measurement in a memory in the toy.

19. The monitoring system of claim 18, wherein the representation of the heart rate measurement is provided in a form of visual data.

20. The monitoring system of claim 18, wherein the representation of the heart rate measurement is provided in a form of audio data.
